(19) United States
(12) Patent Application Publication
(10) Pub. No.: US 2005/0216027 A1
(43) Pub. Date: Sep. 29, 2005

(54) Extraction screwdriver

(76) Inventors: Sean S. Suh, Plymouth Meeting, PA (US); David Sawyer Rathbun, Gap, PA (US)

Correspondence Address: JONES DAY, 222 EAST 41ST STREET, NEW YORK, NY, 10017-6702, US

(21) Appl. No.: 10/809,767

(22) Filed: Mar. 24, 2004

(51) Int. Cl. A61B 17/56

(52) U.S. Cl. 606/104

(57) ABSTRACT

The present invention provides a simplified bone fastener removal tool which allows the surgeon to remove bone screws or other bone fasteners from bone, and from bone plates incorporating fastener locking elements. The tool includes an inner shaft which axially engages the bone fastener, a drive shaft which allows the fastener to be rotated using the tool, and an internally threaded outer sleeve which, in combination with the drive shaft, allows for a controlled removal of the fastener from the bone plate and bone. In particular, the internal threads of the outer sleeve engage external threads on the drive shaft, such that rotating the drive shaft while maintaining the outer sleeve fixed causes the drive shaft to translate with respect to the sleeve. Thus, when a fastener is axially engaged with the drive shaft, a controlled removal of the fastener from the bone and bone plate is accomplished simply by rotating the drive shaft with respect to the outer sleeve. No pulling on the fastener is necessary. A method of using the tool is also provided.

### FIELD OF THE INVENTION

The invention is related to a fastener driving and removal tool. More particularly, the invention relates to an improved fastener driving and removal tool for driving and extracting screws used to secure an orthopedic bone plate to bone.

### BACKGROUND OF THE INVENTION

Orthopedic fixation devices such as plates are frequently coupled to bone with fasteners inserted through plate holes. It is known that such fasteners can often be removed with typical screwdrivers and variations of typical screwdrivers. It is also known that securing such fasteners to the bone plate, for example through the use of expanding locking rings, can decrease the incidence of premature screw loosening and back out. It is also known that, to remove such locked fasteners, removal tools having mechanisms to expand locking rings can be used.

Existing removal tools, however, are inadequate to deal with the problem of fasteners that are seated in substandard bone. Such fasteners often may not be removed by simply backing out the screw, because the bone may not be strong enough to support the threads during the back out procedure. In such cases, the screw may simply turn in place within the bone and additional tooling or engaging elements may be required to secure its removal. Axial engagement elements (e.g. a threaded shaft extending from a cannulation in the driver which engages internal threads formed in the screw head or shank) may be used to couple the screw to the removal tool. Such arrangements, while allowing for removal of screws seated in substandard bone, do not allow for controlled removal of such screws, and instead rely on the surgeon to apply sufficient force to remove the screw but not so much force that the screw is ripped from the surrounding bone causing damage to the bone in which the screw is seated. Risk of such damage may be great, due to a relatively high threshold force which maintains the bone screw in even substandard bone. Thus, there exists a need for an extraction tool that axially engages a bone fastener seated in substandard vertebral bone but which also provides for controlled removal of the fastener under such circumstances so as to minimize the chance for damage to the vertebral bone in which the screw is seated. Also, in the case where a fastener is used to attach a bone plate and a locking device such as a locking ring is used to connect the fastener to the plate, there exists a need to provide an extraction tool which disengages the locking ring sufficiently to allow the fastener to be removed from the plate.

### SUMMARY OF THE INVENTION

The invention relates to a fastener driving and removal tool that includes a knob, a handle, a drive shaft, an inner shaft, and an outer sleeve. The inner shaft extends into and engages the head or shank of the fastener. The driver shaft runs longitudinally with and surrounds the inner shaft (except where the inner shaft engages the fastener). The outer sleeve runs longitudinally with and surrounds the driver shaft. The outer sleeve is axially movable with respect to the driver shaft. The outer sleeve contacts and utilizes the plate surface, from which the fastener is being pulled, as a brace, while the fastener is being removed.

The inner shaft may engage the fastener in a number of ways. The inner shaft may be externally threaded to engage the internal threads of the fastener head or shank. The inner shaft may include radially outwardly extending wings or propellers to slide into corresponding cutouts or grooves in the fastener head.

The outer sleeve and driver shaft are axially movable with respect to each other. The outer sleeve and driver shaft may also be rotationally movable with respect to each other. To allow for relative axial and rotational movement, the outer sleeve may have internal threads to engage external threads of the driver shaft.

If a plate with through-holes or bores is being used, fasteners may be secured to the plate with individual locking clips to prevent the screws from backing out in situ. Each fastener may have, at its head, a circumferential groove which the locking clip of the plate can engage. The driver shaft may have a cruciform shape at its end similar to that of a Phillips screwdriver. The “fins” of the Phillips screwdriver may extend radially outward beyond the inner circumference of the groove in the screw head so as to expand the clip sufficiently to allow the screw to be removed from the plate and bone.

A tool is provided, comprising a drive shaft having proximal and distal ends, an intermediate portion, an outer sleeve engaging portion and a length. The tool may have a handle portion associated with the drive shaft proximal end and a fastener engaging portion associated with the drive shaft distal end. The fastener engaging portion may comprise a first surface configured to axially engage a fastener and a second surface configured to rotationally engage the fastener. The tool may further have an outer sleeve associated with the drive shaft intermediate portion and the sleeve may comprise a drive shaft engaging portion. The outer sleeve engaging portion and the drive shaft engaging portion may be configured to coact to allow at least a portion of the drive shaft to translate linearly within the sleeve.

The drive shaft may comprise a cannulated fastener driving portion and an inner shaft portion. At least a portion of the inner shaft portion may be disposed within the fastener driving portion, and the inner shaft portion may be configured to axially engage the fastener. Further, the driving portion may be configured to rotationally engage the fastener.

The fastener driving portion may further comprise a driving sleeve having a distal end comprising a fastener driving end and a bore having an inner surface, and a shaft portion comprising a distal end having a driving sleeve cooperating portion. A cannulation may be provided for receiving the inner shaft portion of the drive shaft, wherein the distal end of the shaft portion is slidably received within the bore of the driving sleeve, and the bore and the driving sleeve cooperating portion are configured such that rotating the inner sleeve rotates the driving sleeve.

The inner shaft further may comprise a radial groove, the shaft portion of the fastener driving portion further may comprises a slot, and the driving sleeve further may comprises a pin bore, such that a pin disposed within the pin bore and extending through the slot to engage the radial groove may fix the inner shaft and the driving sleeve axially with respect to each other. When the inner shaft axially engages the fastener, the driving sleeve may also engage the fastener. The inner shaft portion may be tapered and the cannulated fastener driving portion may be configured to slidingly receive the tapered inner shaft.

The axial fastener-engagement portion may comprise a thread. The first surface of the fastener engaging portion may comprise at least one radial member configured to axially engage a recess in the head of a bone fastener. The first surface may further comprise a plurality of radial members, each of which is configured to axially engage corresponding recesses in a fastener head. Alternatively, the axial fastener-engagement portion may grip the fastener about an outside surface of the fastener head. The sleeve engaging portion and drive shaft engaging portions comprise complementary threads.

The tool may further comprise an inner shaft having a fastener engaging surface at one end, and the drive shaft may further comprise a cannulation configured and sized to accept at least a portion of the inner shaft, so that when the inner shaft is disposed within the cannulation the fastener engaging surface extends distally beyond the distal end of the drive shaft. At least a portion of the sleeve may have a roughened outer surface.

The fastener may be disposed within a fastener hole in a plate, and the fastener hole may be provided with an expandable locking clip configured to engage a portion of the fastener to prevent the fastener from backing out of the fastener hole. The tool may have a fastener engaging portion comprising a locking clip expanding portion, where the locking clip expanding portion is configured to expand the locking clip. The locking clip expanding portion may be configured to expand the locking clip to a dimension greater than an outer diameter of the fastener head.

Alternatively, the locking clip expanding portion may configured to expand the locking clip to a dimension smaller than an outer diameter of the fastener head. At least a portion of the fastener may be configured to expand the locking clip to a dimension substantially equal to the outer diameter of the fastener head when the tool is engaged with the fastener and the tool is operated to remove the fastener from the bone plate.

The tool sleeve may have a distal end configured to engage a bone surface. Alternatively, the sleeve may have a distal end configured to engage a surface of a bone plate. The sleeve may comprise first and second pieces, the first piece configured to threadably engage the sleeve engaging portion of the drive shaft and the second piece comprising an end configured to engage the surface of a bone plate or bone. The first and second pieces may be rotatable with respect to each other.

A bone plate, tool and fastener system may be provided comprising a tool having a drive shaft having proximal and distal ends, an intermediate portion, an outer sleeve engaging portion and a length. The tool may further have a handle portion associated with the drive shaft proximal end, and a fastener engaging portion associated with the drive shaft distal end, the fastener engaging portion comprising a first surface configured to axially engage a fastener and a second surface configured to rotationally engage the fastener. The tool may additionally have an outer sleeve associated with the drive shaft intermediate portion, the sleeve comprising a drive shaft engaging portion, wherein the outer sleeve engaging portion and the drive shaft engaging portion are configured to coact to allow at least a portion of the drive shaft to translate linearly within the sleeve. The tool may further comprise at least one radial member. A fastener may be provided having a radially deformable head and a threaded body. The head may have a circumferential groove for engaging a bone plate locking element and may be configured to receive the radial member to axially engage the tool with the fastener. A bone plate may be provided having at least one bone screw hole, the at least one bone screw hole having a locking element disposed at least partially within the hole and configured to engage at least a portion of the fastener head groove to axially retain the bone screw within the bone screw hole. Thus, when the fastener is retained within the bone screw hole by the locking element and the tool is axially engaged with the fastener, an axial removal force applied to the fastener by the tool may cause the fastener head to radially deform to thereby disengage the fastener from the locking element.

The fastener head may be rendered radially compressible by at least one longitudinal slot disposed in the head. Alternatively, the fastener head may be rendered radially compressible by a hollow portion disposed in the head.

A tool is provided comprising a drive shaft having a fastener engaging end and a sleeve engaging portion. The fastener engaging end may comprise a rotational engagement portion and an axial engagement portion. A sleeve may be disposed about at least a portion of the drive shaft, the sleeve comprising a drive shaft engaging portion. Further, the sleeve engaging portion and the drive shaft engaging portion may comprise complementary threads configured to allow the drive shaft to translate linearly within the sleeve when the drive shaft is rotated relative to the sleeve. The drive shaft may comprise a cannulated fastener driving portion and an inner shaft portion, at least a portion of the inner shaft disposed within the driving portion, the inner shaft portion configured to axially engage a fastener and the driving portion configured to rotationally engage the fastener. The inner shaft portion may be tapered and the cannulated fastener driving portion may be configured to slidingly receive the tapered inner shaft.

The fastener engaging end may further comprising a locking clip expanding portion, and the fastener engaging end of the drive shaft may be configured to engage a fastener disposed within a fastener hole in a plate, the plate having an expandable locking clip disposed within the fastener hole, the clip configured to engage a portion of the fastener to prevent the fastener from backing out of the fastener hole, wherein the fastener engaging end is configured to expand the fastener locking clip when the drive shaft engages the fastener.

The locking clip engaging portion may be configured to expand the locking clip to a dimension greater than an outer diameter of the fastener head. Alternatively, the locking clip engaging portion may be configured to expand the locking clip to a dimension smaller than an outer diameter of the fastener head. Where the tool is configured to expand the clip to a dimension smaller than the outer diameter of the fastener head, an axial removal force applied by the tool may be greater than a fastener locking force of the locking clip.

The tool sleeve may have a distal end configured to engage a bone surface. Alternatively, the sleeve may have a distal end configured to engage a surface of a bone plate. The sleeve may comprise first and second pieces, the first piece configured to threadably engage the sleeve engaging portion of the drive shaft and the second piece comprising an end configured to engage the surface of a bone plate or bone. The second piece may further comprise an inwardly-extending spring element configured to engage an outer surface of the drive shaft to provisionally retain the second piece at a selected location on the drive shaft. The first and second pieces may be rotatable with respect to each other. The rotational engagement and axial engagement portions may comprise a single screw thread element configured to engage and retain at least a portion of a fastener seated in bone.

When the tool is engaged with the fastener and the tool is rotated to remove the fastener from the bone, the rotation may serve to increase engagement of the screw thread element with the fastener.

A method of removing a fastener from a bone and/or plate is provided comprising the steps of: (a) providing a tool having an inner shaft portion, a cannulated drive shaft portion and a sleeve portion, the cannulated drive shaft portion at least partially disposed within the sleeve portion and the inner shaft portion at least partially disposed within the cannulated drive shaft portion; (b) inserting the drive shaft with the head of a bone fastener; (c) axially engaging the inner shaft portion with the bone fastener, the fastener engaged with a bone portion, the fastener further disposed within the bone screw hole of a bone plate; (d) rotationally engaging the inner shaft portion with the bone fastener; (e) engaging one end of the sleeve portion with a surface of the bone plate; and (f) moving the drive shaft and outer sleeve portions with respect to each other to remove the fastener from the bone.

The inner shaft portion may further comprise a threaded distal end configured to engage an internally threaded portion of the fastener. Steps (b), (c) and (d) may be performed substantially simultaneously. The shaft portion may further comprise an externally threaded portion configured to mate with an internally threaded portion of the outer sleeve, wherein step (f) comprises rotating the drive shaft and outer sleeve portions with respect to each other.

### BRIEF DESCRIPTION OF THE DRAWINGS

The features and advantages of the present invention will become more readily apparent from the following detailed description of the invention in which like elements are labeled similarly and in which:

FIGS. 1a and 1b are side and cross-sectional views, respectively, of a first embodiment of the tool;

FIG. 2 illustrates the distal fastener-engaging portion of a first embodiment of the tool of FIG. 1a in use with a fastener, which is engaged by a first embodiment of a locking clip of a bone plate;

FIGS. 3a through 3e are side, sectional, partial side, end and perspective views, respectively, of the handle and drive shaft portions of the tool of FIG. 1a;

FIGS. 4a, 4b and 4c are side, sectional and top views, respectively, of a bone screw for use with the tool of FIG. 1a;

FIGS. 5a and 5b are side views of two embodiments of an inner shaft portion of the tool of FIG. 1a;

FIGS. 6a and 6b are sectional and side views of the outer sleeve portion of the tool of FIG. 1a;

FIGS. 7a and 7b are top and side views of an exemplary bone plate for use with the tool of FIG. 1a;

FIG. 8 is top view of an exemplary locking clip for use with the plate of FIGS. 7a and 7b and the tool of FIG. 1a;

FIGS. 9a through 9e are perspective and sectional views of a second embodiment of a plate and fastener for use with the tool of FIG. 1a; with an alternative drive shaft arrangement.

FIGS. 10a, 10b and 10c are two perspective and one cross sectional view, respectively, of a second embodiment of the tool of FIG. 1;

FIG. 11 is a sectional view of a third embodiment of the tool of FIG. 1;

FIG. 12 is a side sectional view of a fourth embodiment of the tool of FIG. 1.

### DETAILED DESCRIPTION OF THE PREFERRED EMBODIMENTS

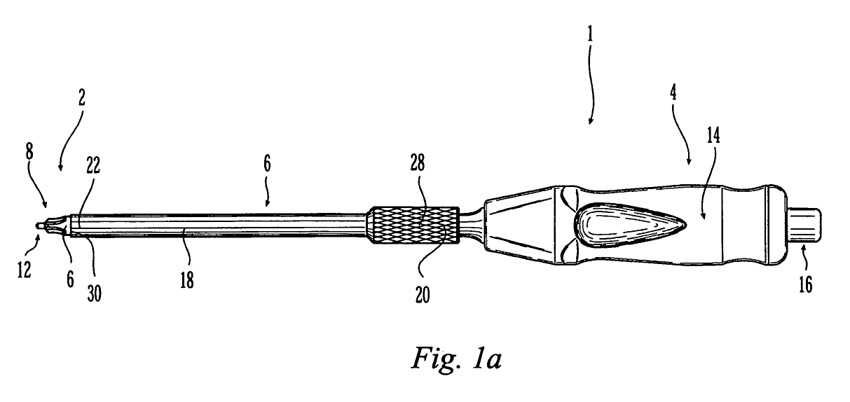

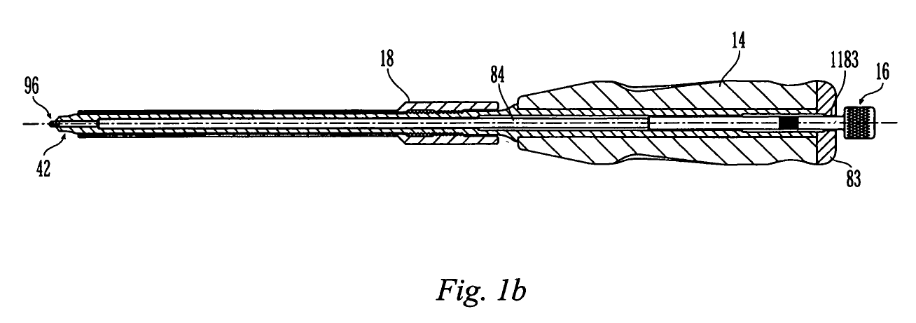

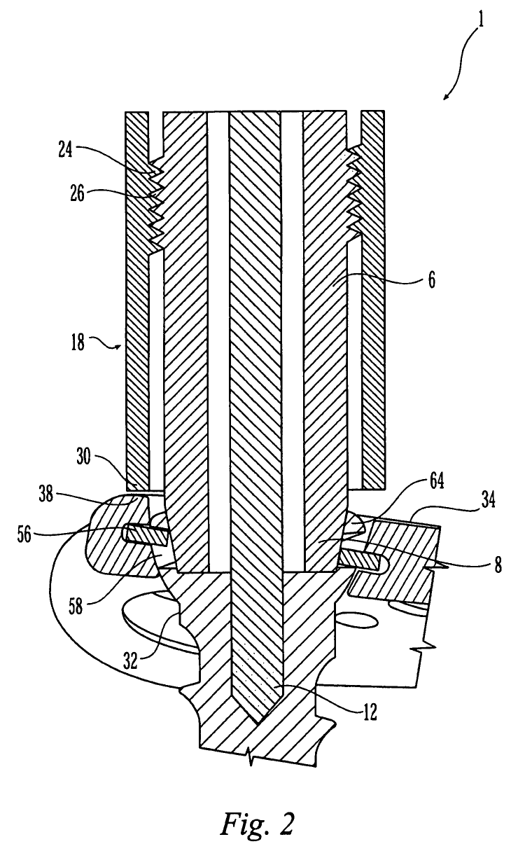

A first embodiment of the fastener driving and removal tool (“the tool”) is shown in FIGS. 1a and 1b. The tool 1 may take the general shape and appearance of a traditional screw driving device, with a distal fastener engaging end 2 and a proximal user end 4, the two ends connected by a drive shaft 6. The fastener engaging end 2 may have a first fastener engaging surface 8 configured and dimensioned to rotationally engage the head of a fastener 32, such as a bone screw (FIG. 2). The fastener engaging end 2 may also have a second fastener engaging surface 12 configured and dimensioned to retain the fastener 32 in fixed axial relation with the tool 1. The user end 4 may comprise a handle 14 configured and dimensioned for gripping by a user. The user end 4 may further comprise an actuator 16 in communication with the second fastener engaging surface 12, thus allowing the user to axially engage the fastener 32 with the tool 1 by actuating the actuator 16. The tool 1 may further comprise an outer sleeve 18 disposed about the drive shaft 6 and extending along at least a portion of the length of the shaft 6. The outer sleeve 18 may have proximal and distal ends 20, 22, and may further comprise an inner translating surface 24 (FIGS. 2 & 6), such as internal threading, for engaging at least a portion of a complementary outer translating surface 26 (FIG. 2) of the drive shaft 6. The two translating surfaces 24, 26 may be configured to allow the outer sleeve 18 to translate along the shaft 6 in response to a user force (e.g. a rotation) applied to an outer gripping surface 28 of the sleeve 18. The distal end 22 of the sleeve 18 may have an abutting surface 30 configured to be engageable with a surface adjacent to the fastener, such as the top surface 38 of a bone plate 34 (FIG. 2) or a bone surface in the case where a bone plate 34 is not used.

The tool 1 may be used to drive a fastener 32 (FIG. 2) into a work piece, such as a bone segment, and it may also be used to remove the fastener 32 from the work piece. When employed to drive a fastener into a work piece, the tool 1 may be used in the manner of a traditional screwdriver, although the user may elect to axially engage the fastener with the tool to ease handling of the fastener by retaining the fastener to the tool. Where the tool 1 is used to remove the fastener 32 from the work piece, the second fastener engaging surface 12 may be engaged with the fastener to axially engage the fastener with the tool 1 to allow for the application of an axial removal force to the fastener in addition to the typical back-out force applied by reverse threading the fastener. As previously noted, this axial engagement feature is particularly useful when the fastener to be removed is seated in substandard bone, because such bone may provide insufficient structural support for the fastener threads and thus the fastener may simply spin in place if a simple reverse threading motion is applied. With the axial engagement feature applied, the user may simply pull up on the tool to remove the fastener from the bone and bone plate. Alternatively, the outer sleeve 18 may be used to draw the drive shaft 6 and the fastener 32 up and out of the bone and the bone plate 34 by holding the outer sleeve rotationally steady while turning the tool handle counter-clockwise. Rotating the drive shaft 6 with respect to the outer sleeve 18 causes the drive shaft (and the attached fastener 32) to translate along the outer sleeve, pulling them away from the bone and bone plate.

Referring to FIG. 2, when the tool 1 is employed to remove a fastener such as a bone screw 32 from, for example, a bone plate 34 that has been screwed to a vertebral body (not shown), the user may also employ the outer sleeve 18 to provide a controlled axial removal force to the bone screw 32. To operate the tool in this way, the user may engage the screw head 32 with the first and second screw engaging surfaces 8, 12 of the tool 1, and lock the screw 32 axially to the tool 1 by rotating the actuator 16. The user then may manually translate the outer sleeve 18 along the drive shaft 6 until the abutting surface 30 of the sleeve 18 engages the top surface 38 of the bone plate 34 (FIG. 2). Thereafter, the user may use one hand to maintain the outer sleeve 18 rotationally fixed while the other hand grips the handle 14 to apply a reverse rotation to the drive shaft 6 to back the screw 32 out of the bone. The reverse rotation of the drive shaft 6 serves to linearly translate the drive shaft 6 with respect to the outer sleeve 18 so that the drive shaft 6 and the attached screw 32 together are drawn axially out of the bone and the bone plate 34. Thus, removal of the bone screw 32 from the bone and the bone plate 34 may be carefully controlled by the user who controls rotation of the sleeve 18 with respect to the drive shaft 6. In this way, the chance for over-force removal of the bone screw 32 is eliminated, and the attendant damage to the surrounding vertebral bone is likewise minimized.

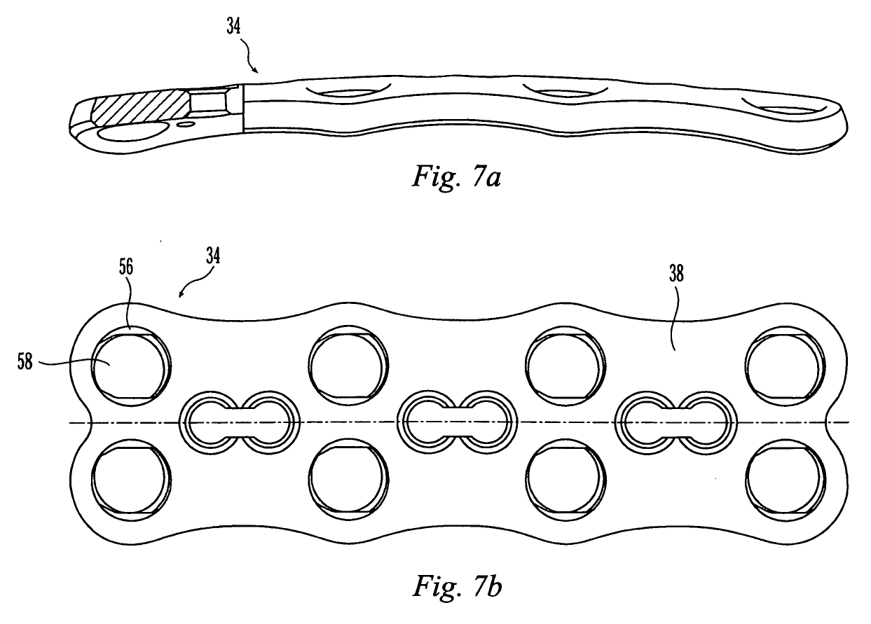

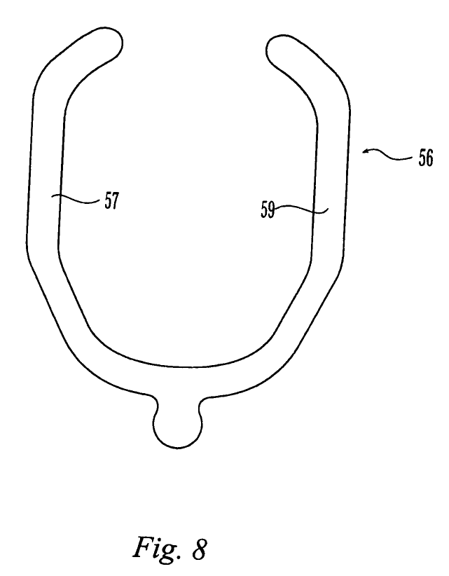

In one embodiment, the tool 1 may comprise a surface 51 capable of expanding a locking clip 56 disposed in a screw hole 58 of the bone plate 34. Such a clip 56 (FIG. 8) may have a portion that extends into the bone screw hole 15 (FIG. 7b) to engage a groove 62 in the head of the bone screw 32 so as to prevent premature back-out of the screw in situ. In use, the locking clip expanding surface 51 may at least partially expand the locking clip 56 when the drive shaft 6 engages the head of the screw 32 so that the bone screw 32 may thereafter be removed from the bone screw hole 15.

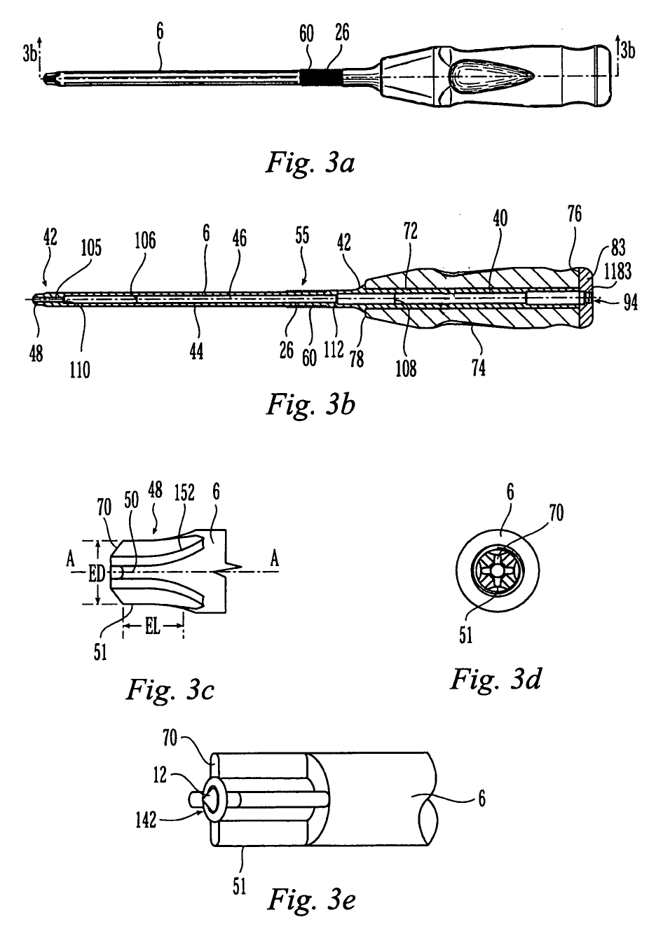

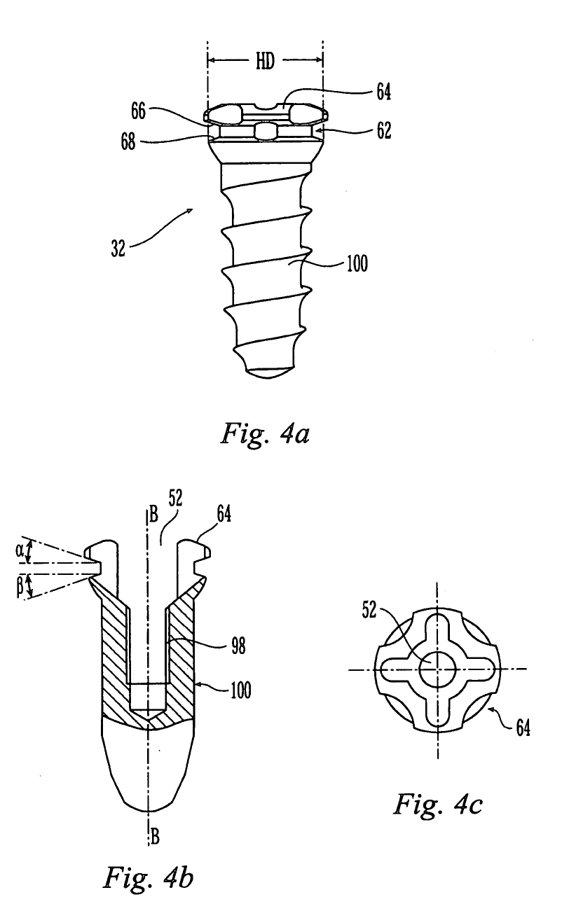

Details of the individual tool elements are shown more particularly in FIGS. 3-6. Drive shaft 6, illustrated in FIGS. 3a-3e, may comprise a proximal handle engaging portion 40 and a distal fastener engaging portion 42, with a cylindrical shaft portion 44 comprising a cannulation 46 connecting the two. The distal fastener engaging portion 42 may comprise a first fastener engaging surface 48, which may further comprise a fastener recess engaging surface 50 and a locking clip expanding surface 51 The first fastener engaging surface 48 may comprise any appropriate fastener engaging geometry, such as a flat or Phillips-style configuration, or it may comprise a socket end such as a hex socket. In the illustrated embodiment, the first fastener engaging surface 48 comprises four blades each having a reduced-cross section portion 50 dimensioned to engage a corresponding recess 52 in the head of a fastener such as a bone screw 32 (FIG. 4b, c). At least a portion of the first fastener engaging surface 48 may comprise a locking clip expanding surface 51, at least a portion of which may be oriented substantially parallel to the longitudinal axis “A-A” of the drive shaft 6 thus resulting in a length “EL” having a constant cross-sectional “blade width” “ED” when the shaft 6 is rotated. When used to remove a bone screw 32 from a bone plate 34 having a locking clip 56 (FIG. 8) disposed in a bone screw hole 58 (FIG. 7b) and configured to retain the head of the screw 32, it is this “blade width” “ED” that expands the locking clip 56 while the bone screw 32 is being removed from the bone plate 34. The length “EL” and “blade width” “ED” may be sized such that the locking clip expanding surface 51 expands the locking clip 56 sufficiently to allow the bone screw 32 to be removed from the bone plate 34. In one embodiment, the “blade width” “ED” is sized so that the locking clip expanding surface 54 may expand the locking clip 56 to a diameter greater than a head diameter “HD” (FIG. 4a) of the bone screw 32. In an alternative embodiment, the “blade width” “ED” is sized so that the locking clip expanding surface 51 may expand the locking clip 56 by an amount smaller than diameter “HD” of the bone screw 32 (in which case a portion of the fastener head (e.g. lower locking clip engaging surface 68) may act to expand the clip to head diameter “HD” as the screw is backed out of the screw hole). The lower locking clip engaging surface 68 may be configured to expand the locking clip 56 only after the clip has been partially expanded by the clip expanding surface 51 of drive shaft 6. In an alternative embodiment, lower locking clip engaging surface 68 may be configured to expand the locking clip 56 without any prior expansion of the clip by the drive shaft 6. In such a configuration, the clip engaging surface 68 of the screw may not expand the clip when the screw is exposed to normal in situ forces, but may expand the clip when sufficient extraction force is applied via the tool 6.

FIG. 3e illustrates an alternative embodiment of the distal fastener engaging portion 42 of FIG. 3c. The fastener engaging portion 142 of FIG. 3e may comprise the same basic fastener engaging elements as described in relation to the embodiment of FIG. 3c, with the difference being that fastener engaging portion 142 is formed by milling, and thus does not have the flared surface 151 of the FIG. 3c embodiment, which is present in the FIG. 3c embodiment due to the geometry of the machining wheel used to form the distal end of the drive shaft.

The drive shaft 6 may further comprise an intermediate portion 55 located between the distal fastener engaging portion 44 and the proximal handle engaging portion 42. The intermediate portion 55 may further comprise the translating surface 26 which, in one embodiment is an externally threaded region 60 for engaging a corresponding portion of the outer sleeve 18 (discussed more in detail below).

An exemplary fastener is shown in FIGS. 4a-b the fastener is a bone screw 32 having a circumferential locking clip groove 62 formed in the fastener head 64. The locking clip groove 62 may comprise upper and lower clip engaging surfaces 66, 68, each of which comprises a surface angle, α, β. In one embodiment, at least the lower locking clip engaging surface 68 has a surface angle β that is not orthogonal with respect to the fastener longitudinal axis “B-B.” Where the fastener 32 has a lower locking clip engaging surface 68 with such a non-orthogonal surface angle β, the locking clip groove 62 itself may apply a small expansion force to the locking clip 56 (owing to the small radial component of the lower groove surface angle) when the fastener 32 is being backed out of the bone. This expansion force may not be sufficient to expand the locking clip 56 when the clip is fully engaged with the groove 62, however, when the clip is expanded slightly using the locking clip expanding surface 51 of the tool, subsequent expansion force applied by the lower surface of the locking clip groove 62 may be sufficient to complete the expansion of the clip 56 so that the bone screw 32 may be removed from the plate 34. Alternatively, the angle β may be chosen so that the expansion force applied by the lower surface of the locking clip groove 62 is sufficient to expand the clip 56 without any prior expansion by the tool. Such arrangements may allow the use of a tool having a smaller fastener engaging surface “blade width” “ED,” thus allowing a smaller recess in the bone screw head 64, thereby increasing the strength of the screw head 64.

The fastener engaging portion may further comprise a beveled tip 70 (FIG. 3c) to facilitate alignment and engagement of the tool 1 with the fastener head recess 52 (FIG. 4b).

The tool handle 14 may comprise a drive shaft engaging portion 72 a gripping portion 74, and proximal and distal ends 76, 78. The drive shaft engaging portion 72 may comprise a cylindrically hollow interior space 80 configured to receive the drive shaft proximal handle engaging portion 40. The handle proximal end 76 may engage a raised annular region of the drive shaft 42, and the handle distal end 78 may engage a flanged proximal region 83 of the drive shaft 6, the two regions of the drive shaft, 42, 83 acting as abutting surfaces to thereby capture the handle 14 and maintain its axial position on the drive shaft. The handle gripping portion 74 may comprise any appropriate ergonometric surface configuration known in the art, and it may be manufactured of any appropriate material known in the art, such as wood, phenolic resin, etc. In one embodiment, the handle may be manufactured from a silicone material to provide enhanced user-feel and grip-ability.

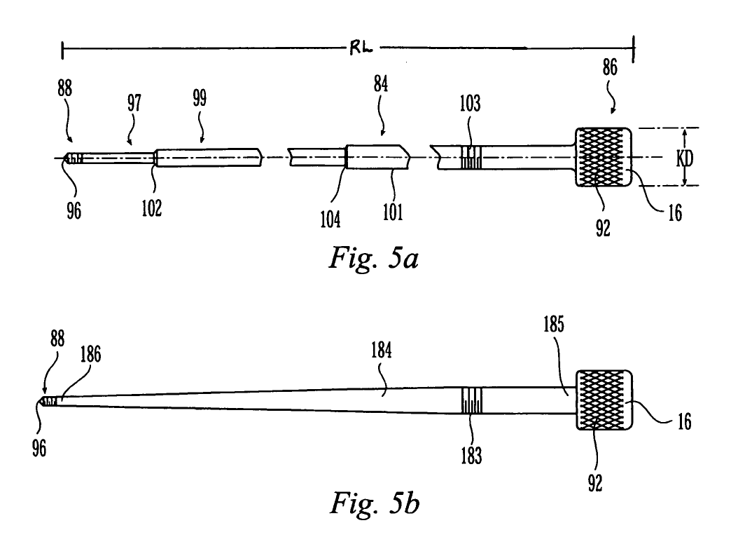

As shown in FIG. 3b, the drive shaft 6 may comprise a cannulation 46 sized and configured to slidingly accept an inner shaft 84 (FIG. 5a) which may be used to axially engage a fastener such as a bone screw 32. The inner shaft 84 may have proximal and distal ends 86, 88, and a length “RL,” (FIG. 5a). The shaft proximal end 86 may comprise an actuator knob 16 having an increased diameter “KD” (FIG. 5a) compared to that of the shaft. The actuator knob 16 may comprise a gripping surface 92, which in the illustrated embodiment is a knurled surface. The rod length “RL” may be such that when the shaft 84 is fully inserted into the drive shaft cannula 46 beginning at the drive shaft proximal end 94, the actuator knob 16 may abut the drive shaft proximal end flange portion 83 while allowing a portion of the inner shaft distal end 88 to extend out from the distal fastener engaging portion 44 of the drive shaft 6. The portion of the inner shaft distal end 88 that extends beyond the distal fastener engaging portion 42 of the drive shaft 6 may comprise a fastener engaging portion 96, configured to engage corresponding internal threads 98 formed in the head 64 or shank 100 of the bone screw 32 to be engaged. In the illustrated embodiment, the fastener engaging portion 96 comprises external threads. However, other appropriate fastener engaging configurations may be used, as will be discussed in more detail below.

The inner shaft 84 may be cylindrical and may be sized to slide axially and rotationally within the drive shaft cannulation 46. The inner shaft 84 may comprise different diameter portions 97, 99, 101, and the transitions between such portions may form external shoulder regions 102, 104. The portions 99, 101 may be sized to correspond to different internal diameter portions 105, 106, 108 of the drive shaft cannulation 46, and the transitions in the cannulation portions may form internal shoulder regions 110, 112 which correspond with the shoulder regions 102, 104 of the inner shaft. These corresponding shoulder regions 102, 104, 110, 112 may cooperate to maintain the axial position of the inner shaft 84 within the cannulation 46 to prevent binding of the actuator knob 16 with the drive shaft proximal end flange 83 when the fastener 32 is fully engaged with the inner shaft 84. FIG. 5b shows an alternative embodiment of inner shaft 84 in which the shaft portion 184 is tapered, having a larger diameter portion 185 located adjacent the actuator knob 16 and a smaller diameter portion 186 located adjacent fastener engaging tip 96. In the illustrated embodiment, the taper in shaft 184 begins adjacent the fastener engaging tip 96 and ends at threaded region 183. Threaded region 183 is thus cylindrical, as is shaft portion 185. In a further alternative embodiment (not shown), the shaft portion may comprise a series of tapered portions, with each portion having a different taper degree. The differing shaft diameters, as well as the degree of each shaft taper, may be provided in any appropriate combination, as will be appreciated by one of skill in the art.

The inner shafts 84, 184 may each further comprise an axial retention feature in the form of an externally threaded region 103, 183 configured to threadably engage an internally threaded portion 1183 of the flanged proximal region 83 of the drive shaft 6 (FIG. 3b). This axial retention feature ensures that when the inner shaft 84, 184 is fit within the drive shaft cannulation 46, the inner shaft 84, 184 will not inadvertently slide off the end of the drive shaft 6. During assembly, the inner shaft 84, 184 is slid into the cannulation 46 until the externally threaded region 103, 183 of the inner shaft 84, 184 engages the inner threading 1183 of the drive shaft proximal region 83. The inner shaft 84, 184 is then rotated so that the threaded sections engage one another, and rotation is continued until the threaded region 103, 183 completely pass through the drive shaft threading 1183 such that the threaded region 103, 183 resides in an unthreaded portion of the drive shaft cannulation 46. The inner shaft 84, 184 is thus loosely axially retained within this portion 46 of the drive shaft 6, and is prevented from sliding proximally out of the drive shaft by the axial interference between the threaded sections 103/183, and 1183. The inner shaft 84, 184 remains free to rotate within the drive shaft 6 to facilitate engaging a fastener at the inner shaft distal end 96.

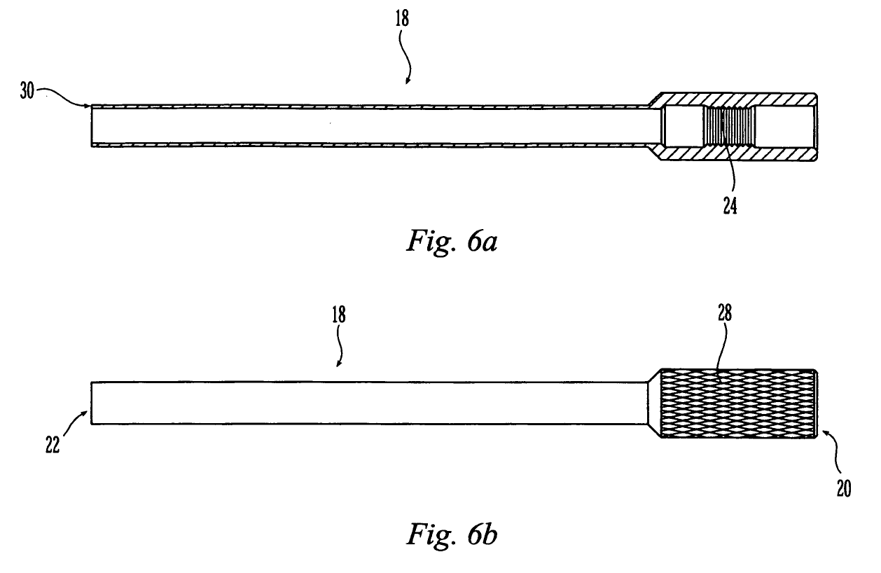

As shown in FIG. 6, the tool 1 may comprise an outer sleeve 18, as previously noted. The outer sleeve 18 may generally comprise a cylindrical sleeve with proximal and distal ends 20, 22. The proximal end 20 may comprise an outer gripping surface 28 and an inner translating surface 24, which in this embodiment comprises threads. It is noted that although a threaded surface is shown, translating surface 24 may comprise any translating arrangement known in the art, such as a ratchet and release mechanism in which a series of ratchet teeth may be disposed on the inner translating surfaces and may be configured to cooperate with a pawl/release mechanism integrated into the outer translating surface 26. The outer gripping surface 28 may be configured to facilitate gripping by the user to allow rotation of the sleeve 18 in use. In the illustrated embodiment, this gripping surface 28 comprises a knurled configuration, though any appropriate surface finish may be provided. The inner translating surface 24 may comprise threads that correspond to threads of the outer translating surface 26 of the drive shaft 6. The threads of the outer sleeve 18 and the drive shaft 24, 26 may cooperate to allow the sleeve 18 to translate along the drive shaft 6 when the sleeve is rotated by the user. Alternatively, the drive shaft 6 may translate along the sleeve 18 when the sleeve is held fixed and the drive shaft is rotated. The sleeve distal end 22 may comprise an abutting surface 30 configured to engage the top surface 38 of a bone plate 34 or the bone itself where the fastener is not used in conjunction with a plate.

FIG. 2 shows the tool 1 engaged with a fastener 32, the fastener 32 engaged with the plate 34 and a plate locking clip 56. The illustrated fastener is a bone screw 32 having a head portion 64 with a recess 52, an externally threaded outer shank portion 100, and an internally threaded inner shank portion 98. The threaded inner shank portion 98 is shown engaged with the threaded distal end 88 of the inner shaft 84. It is noted that although the illustrated embodiment shows the inner threads as being disposed within the fastener shank 100, the threads could alternatively be disposed in the fastener head, or they could be disposed in both the head and the shank.

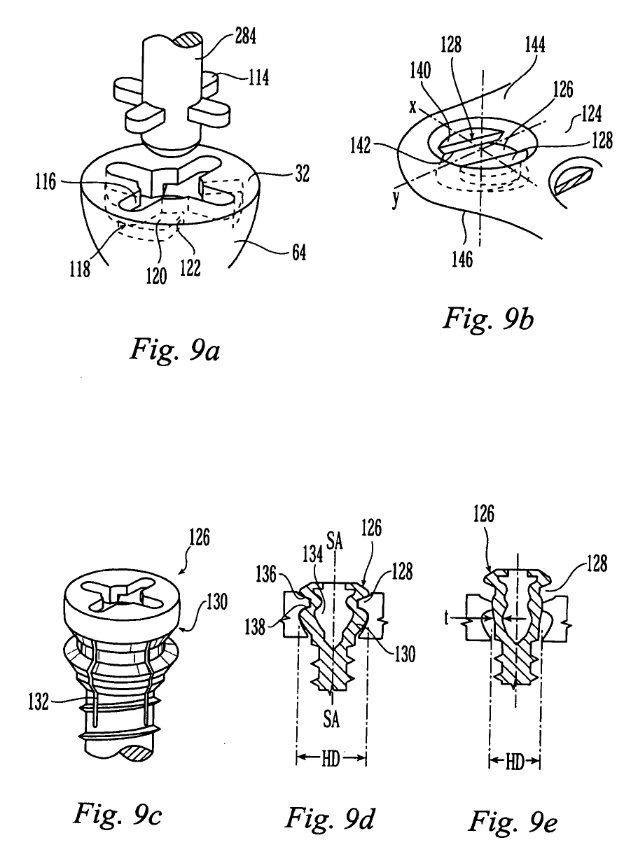

Likewise, while the illustrated embodiment shows the inner shaft 84 and fastener 32 being threadably connected, any other suitable engagement configuration may be employed to axially lock the tool and fastener together. Such configurations could comprise a friction fit between the shaft and fastener using corresponding or mismatched tapered surfaces. Alternatively, an external coupling element may be provided to engage the outside surface of the fastener head. A further suitable connection arrangement could be that described in relation to FIG. 9a below, in which a plurality of radial protrusions on the inner shaft or drive shaft may be axially retained by corresponding recesses formed in the fastener head.

FIG. 2 also illustrates the interaction between the translating surfaces 26, 24 of the outer sleeve 18 and the drive shaft. 6 In the illustrated embodiment, the translating surfaces comprise complementary threads. As shown the outer sleeve 18 is positioned so that its distal end abutting surface 30 lies adjacent to the top surface 38 of the bone plate 34. In this position, any further rotation of the outer sleeve 18 with respect to the drive shaft (or conversely—rotation of the drive shaft within the sleeve) may cause the sleeve distal end to contact the bone plate 34. Thereafter, further rotation of the outer sleeve 18 with respect to the drive shaft may cause the drive shaft to be drawn up into the sleeve along with the fastener, withdrawing the fastener from the plate and the underlying bone. Since the outer sleeve 18 firmly abuts the top surface 38 of the bone plate 34, the removal forces imparted on the fastener via the inner shaft 84 are transmitted directly to the bone plate 34, without relying on the underlying bone to support the screw threads during back-out. Thus, a controlled removal of the screw from the plate and bone may be achieved.

FIGS. 7a-b show an exemplary bone plate 34 and locking clip 56 arrangement, in which a single locking clip 56 is disposed in each screw hole 14 of the plate 34 so that the clip lies within a circumferential groove (FIG. 2) in the bone plate 34. FIG. 8 illustrates an exemplary locking clip 56 that may be used to retain a fastener within the bone plate 34. The clip comprises a single unitary piece having first and second locking legs 57, 59 which, when installed in the bone plate 34, may engage the cylindrical groove 62 in the fastener head 64 when the fastener 32 is installed in the plate 34.

To extract a fastener 32 from a bone plate 34, the fastener engaging end 2 of the tool 1 may be aligned with the corresponding recess 52 in the fastener head 64, and distal end 88 of the inner shaft 84 may be inserted into the fastener head 64. The actuator knob 16 may then be turned in a first direction to connect the threaded inner shaft 84 with the corresponding internally threaded area 100 of the fastener 32, thus drawing the tool and fastener into tight engagement. Tightening the actuator knob 16 in this manner causes the fastener 32 to be axially and rotationally locked to the tool 1. The outer sleeve 18 may then be rotated about the drive shaft 6 so that the corresponding threaded surfaces 24, 26 cause it to translate along the shaft toward the plate 34, stopping when the sleeve abutment end 30 contacts the plate's top surface 38. Then, holding the outer sleeve 18 stationary by holding the gripping surface 28, the user may turn the handle 14 to rotate the drive shaft 6 in the direction required to back the fastener 32 out of the bone and plate. This rotation of the drive shaft 6 also causes the drive shaft 6 to translate up into the outer sleeve 18, carrying the axially engaged fastener 32 with it, up and out of the bone plate 34 and the underlying bone. Once removed from the plate 34, the fastener 32 may then be removed from the tool 1 by reverse-rotating the actuation knob 16, which disengages the corresponding threads 96, 98 of the inner shaft 84 and the fastener 32.

When removing a fastener 32 from a bone plate system that utilizes a locking clip 56 disposed in the fastener hole 58 to retain the head 64 of the fastener 32, the clip engaging surface 51 of the tool 1 (FIG. 3c) may expand the locking clip 56 when the tool 1 engages with the fastener 32, so that the fastener 32 may be removed from the bone and bone plate. As noted previously, the tool may be configured to expand the clip only slightly (i.e. the partially expanded clip may still engage at least a portion of the screw head groove 62), or the tool may be configured to expand the clip to a dimension greater than head diameter “HD” (FIG. 4a). A portion of the screw head (e.g. lower locking clip engaging surface 68, may also contribute to the expansion of the clip 56 so that the associated bone screw 32 may be easily removed from its bone screw hole.

As shown in FIGS. 9a-9d, an alternative embodiment of the axial engagement mechanism between the drive shaft 284 and the fastener 32 may be provided. In this embodiment, the drive shaft 284 is non-cannulated and does not have an inner shaft portion. Drive shaft 284 may comprise one or more radially extending wings 114, and the fastener head 64 may comprise a corresponding number of interconnected recessed portions 116 of corresponding shape configured to receive the wings 114. In the illustrated embodiment, the drive shaft 284 comprises four such wings 114, and the fastener head 64 comprises four corresponding and interconnected recessed portions 116. Each recessed portion 116 may further comprise a circumferentially extending interlock pocket 118 configured to accept a corresponding wing 114 when the wing is inserted into the recess 116 and rotated counter-clockwise The interlock pocket 118 may have an overhang surface 120 which prevents the wing 114 from being axially removed from the pocket, thus axially locking the drive shaft 84 to the fastener 34. This embodiment may provide a simplified engagement arrangement and procedure as compared to the threaded engagement mechanism described in relation to FIG. 2, since it requires only a small degree of rotation of the actuator knob 16 in order to lock the tool 1 to the fastener 32. It also reduces the complexity of the drive shaft arrangement, since the drive shaft of this embodiment comprises only a single piece. The “winged” arrangement also eliminates any chance of cross-threading between the drive shaft 284 and the fastener which could result in damage to the threads and attendant difficulty in removing the fastener from the bone and plate.

The wing arrangement of FIG. 9a may be used with any of the previously described fastener, plate and locking clip arrangements, or it may be used with the alternative screw and plate locking mechanism shown in FIG. 9b-9e. FIG. 9b shows a plate 124 comprising at least one bone screw hole 126, the bone screw hole 126 comprising a pair of opposed locking gussets 128. The locking gussets 128 may be rigid or they may be at least partially flexible. The plate 124 of this embodiment may be used with fastener 126 having a circumferential locking groove 128 disposed in its head 130 configured to receive the locking gussets 128. The fastener 126 of this embodiment may further be configured so that its head 130 is radially compressible. Thus, during installation and removal the fastener 126, the fastener head itself flexes (see FIG. 9e), while the gusset 128 remains rigid. To impart the desired flexibility, the fastener head 130 may comprise at least one longitudinally disposed slot 132, which allow the head 130 to contract upon installation and removal from the plate 124. Thus, when the fastener is placed in the bone screw hole and driven into the underlying bone, the head may radially contract (FIG. 9e) so that the diameter “HD” of the lower portion of the fastener head is equal to or smaller than the lateral distance between the gussets 128, thus allowing the lower portion of the fastener head 130 to clear the gussets 128. A similar radial contraction may occur when the fastener is removed from the plate and bone. To provide even greater flexibility, the fastener head 130 may further comprise a hollow center portion 134 configured to reduce the wall thickness “t” (FIG. 9e) of the head region, and thus further reducing the amount of force required to cause the head to flex upon installation and removal of the fastener 126 from the plate.

The fastener 126 of FIGS. 9a-9e further may comprise upper and lower gusset contacting surfaces 136, 138 which may be non-orthogonal to the longitudinal axis “SA-SA” of the screw. The gusset upper and lower surfaces 140, 142 likewise may be oriented non-orthogonal to the plate upper and lower surfaces 144, 146. In the illustrated embodiment, the fastener upper and lower gusset contacting surfaces 136, 138 may be angled such that together they form a “V” shape, and the gusset upper and lower surfaces 140, 142 may likewise be so angled. This angled configuration of the gussets and gusset contacting surfaces may facilitate contraction of the flexible fastener head 130 during installation and removal of the fastener with the plate 124.

The dimensions and arrangements of the slots 132 and center cutout portion 134 may be configured in any appropriate manner so as to provide a flexible fastener head 126 that easily contracts when driven into and removed from the plate 124, while also providing a sufficiently stiff structure to resist premature back-out in situ. Likewise, the angles of the upper and lower gusset contacting surfaces 136, 138, as well as the gusset upper and lower surfaces 140, 142 themselves, may be selected to hinder premature back-out but to ease installation and removal of the fastener in the plate. It is noted that, while this embodiment is illustrated as having an axial engagement mechanism utilizing wings 114 on the inner shaft 84 and corresponding recesses 116 and interlock pockets 118, a threaded connection similar to that described for previous embodiments could also be used, simply by providing threads within the screw shank and providing an inner shaft 84 having sufficient length to reach the inner shank threads.

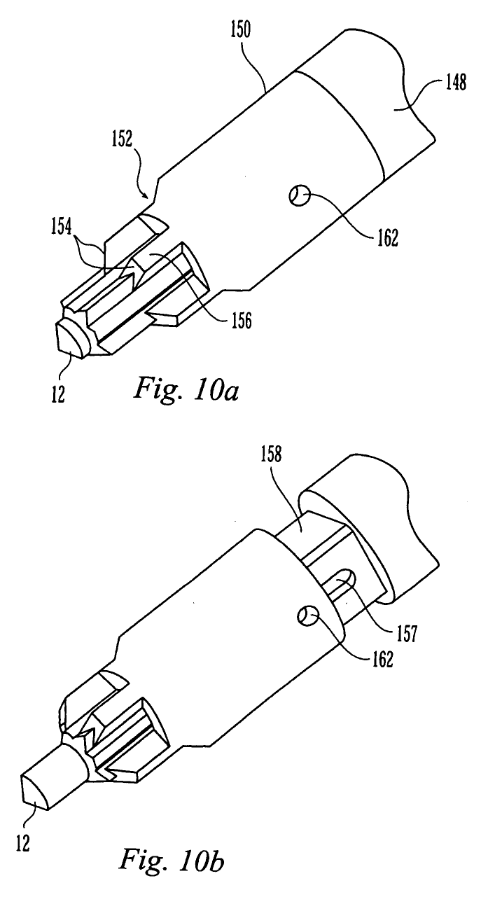

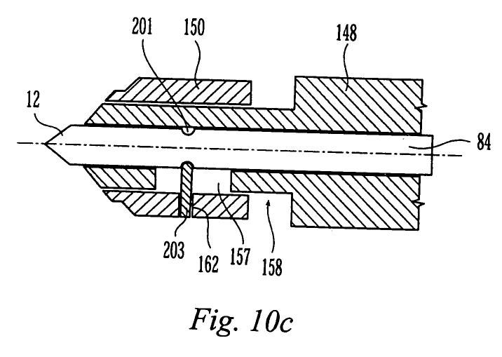

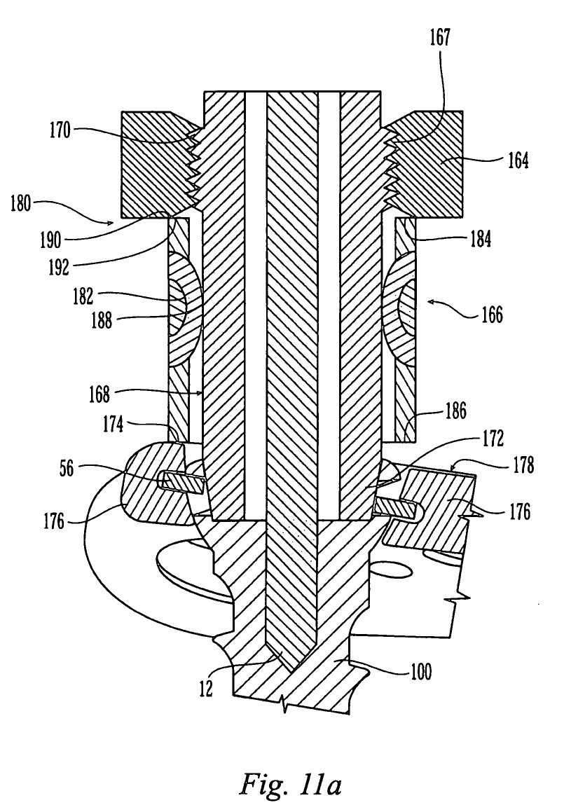

As shown in FIGS. 10a and 10b, yet another embodiment the tool 1 may comprise a drive shaft 148 having a separate drive sleeve 150 located at the shaft distal end 152, the sleeve 150 being configured both to disengage a fastener locking clip (where the fastener is used with a plating system that incorporates individual fastener locking clips), and to rotate the fastener 32. The drive sleeve 150 may comprise at least one fastener recess engaging surface 154 and a locking clip expanding surface 156 similar in all respects to like titled surfaces described in relation to the embodiment of FIG. 1a, except that the two surfaces of the present embodiment are located on a sleeve element 150 separate from the remainder of the drive shaft 148.

The drive shaft 148 may have a reduced cross-section portion 158, and the drive sleeve 150 may have an inner bore 160 configured to slidingly engage the drive shaft reduced cross-section portion 158. The drive sleeve inner bore 160 and the drive shaft reduced cross-section portion 158 may have corresponding non-circular cross-sections to allow the transmission of torque between the pieces, thus allowing the drive sleeve 150 to drive the fastener 32 upon rotation of the tool handle 14. In one embodiment, the corresponding cross-sections may be generally square, although other non-circular geometric shapes may also be used.

In order to allow the locking clip expanding surface 156 to engage and at least partially expand the locking clip 56 when the tool 1 is engaged with the fastener 32, the drive sleeve 150 may be pinned to a radial groove 201 formed in the inner shaft 84 (FIG. 10c). Specifically, pin 203 may be fixed to a bore 162 in the drive sleeve 150 and may project radially inward through window 157 formed in drive sleeve 158 to engage with radial groove 201. Thus, the pin 203 may be axially fixed to inner shaft 84, but the inner shaft 84 may rotate with respect to the pin 203. This arrangement allows the drive sleeve 150 to move into engagement with the fastener head as the inner shaft 84 is threaded into the fastener 32, thus, the locking clip 56 may be at least partially expanded as the fastener 32 is threaded onto the rod 84. To facilitate engagement of the pin 203 between the drive sleeve 150 and the inner shaft 84, a longitudinal window 157 may be provided in reduced cross-section portion 158 of the drive shaft 148.

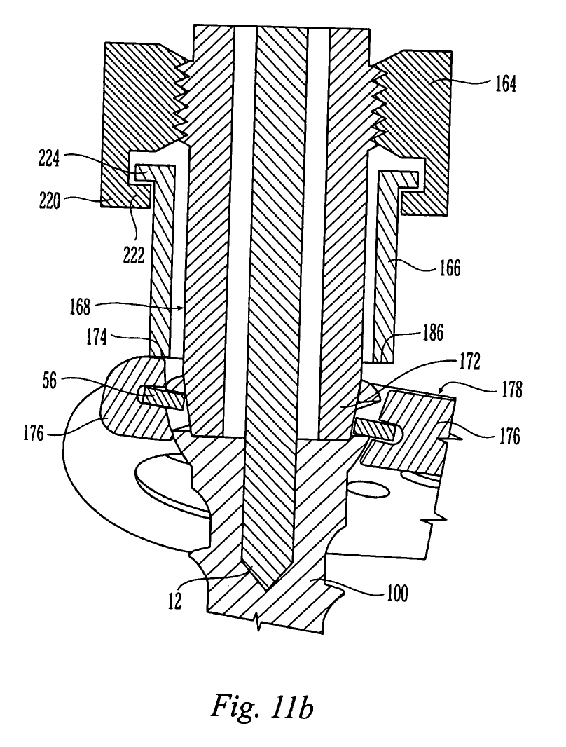

A further embodiment of the tool outer sleeve is illustrated in FIG. 11a, in which the outer sleeve is formed in two pieces comprising a nut portion 164 and a spring-sleeve portion 166. The nut portion 164 comprises internal threads 167 and is disposed about the drive shaft 168 to engage a correspondingly threaded portion 170 of the drive shaft. The spring-sleeve portion 166 is disposed about the drive shaft 168 between the nut portion 164 and the fastener engaging portion 172 of the drive shaft and comprises an abutting surface 174 for engagement with a bone plate 176. The tool of this embodiment functions similarly to the previously described embodiments in which a single piece outer sleeve 18 is provided. This embodiment, however, the two-piece design of the present embodiment provides the added advantage that it allows the abutting surface 174 of the spring sleeve portion 166 to remain rotationally stationary with respect to the top surface 178 of the bone plate 176 (FIG. 11) throughout the screw extraction procedure. This is because all of the rotational movement necessary to facilitate the translation of the drive shaft 168 and the sleeve 166 occurs at the interface 180 between the nut 164 and the spring sleeve 166, so that the spring sleeve 166 may remain rotationally fixed.

The spring-sleeve 166 may further comprise a spring element 182, disposed between the sleeve's proximal and distal ends 184, 186. This spring element 182 may resiliently engage the outer surface 188 of the drive shaft 168 to provide provisional fixation of the spring-sleeve 166 along the drive shaft to ensure the spring-sleeve does not slide off the end of the drive shaft.

The nut and spring-sleeve may have cooperating end surfaces 190, 192 configured to axially retain the two pieces, while still allowing the nut 164 to rotate with respect to the spring sleeve 166. FIG. 11b shows an alternative embodiment in which the nut end portion 220 may comprise a radially-extending collar 222 and the sleeve 166 may comprise a radially-extending lip 224-, the collar and lip 222, 224 coacting to axially fix the two pieces, while still allowing them to rotate with respect to each other. Providing this axial retention mechanism may obviate the spring element 182 (FIG. 11a), which may be eliminated as the nut 164 itself will serve to retain the sleeve 166 on the tool.

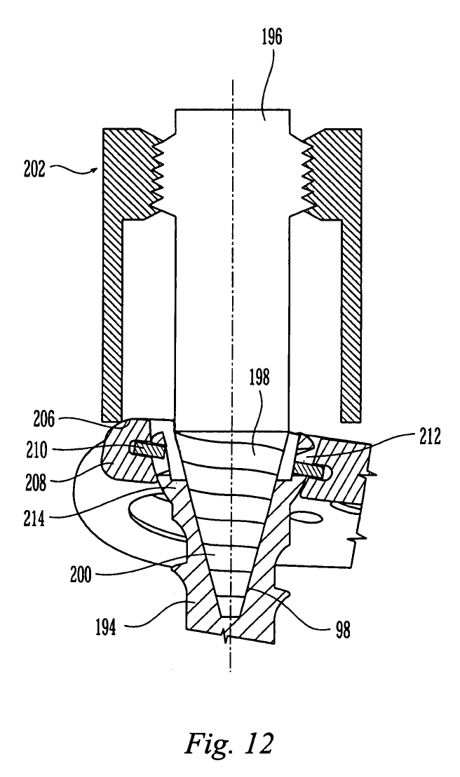

FIG. 12 illustrates another embodiment of the tool which may be used to remove a fastener 194 having a damaged internal engagement thread 98. The tool of this embodiment may be used where the internal engagement thread 98 is damaged in situ, or becomes damaged due to cross-threading with the threaded engaging end 96 of the inner shaft 84, or other occurrence such that the user is unable to engage the fastener with the inner shaft 84. The tool of this embodiment may comprise an extraction shaft 196 in lieu of the drive shaft and inner shaft of previous embodiments. The extraction shaft 196 may have a conical thread 198 at its distal end 200, and the thread 198 may be of a “reverse hand” such that the normal reverse rotation applied to back out the fastener may serve to dig the conical thread 198 into the damaged fastener threads 98 (thus axially locking the tool to the fastener) and also to back the fastener 194 out of the bone and plate. The tool of this embodiment may further comprise an outer sleeve 202 arranged coaxially about the extraction shaft 196, and the outer sleeve 202 may operate in the same manner as described previously in relation to other embodiments. Thus the extraction shaft 196 may act to engage the fastener, while the outer sleeve may act to allow a controlled removal of the damaged fastener by transferring the removal force to the top surface 206 of the bone plate 208. As with the previous embodiment, the outer sleeve may be either one or two pieces.

Where the fastener is used with a plating system having a locking clip 210 disposed in each screw hole 212 to retain the head 214 of the fastener 194, a locking clip expansion sleeve 218, separate from the extraction shaft 196, may be provided to allow the damaged screw 216 to be removed from the plate unimpeded by the clip 210. The locking clip expansion sleeve of this embodiment may be used solely to expand the locking clip 210, or it may comprise drive elements 218 configured to engage the drive recesses on the fastener such that rotation of the extraction shaft 196 may cause the locking clip expansion sleeve 218 to rotate the screw 216.

The individual elements of the embodiments of the tool may be formed using any appropriate method known in the art. Likewise, the elements may be made of any appropriate material or combination of materials, including stainless steel, aluminum, titanium, polymers, etc.

Accordingly, it should be understood that the embodiments disclosed herein are merely illustrative of the principles of the invention. Various other modifications may be made by those skilled in the art which will embody the principles of the invention and fall within the spirit and the scope thereof.

1. A tool comprising:
a drive shaft having proximal and distal ends, an intermediate portion, an outer sleeve engaging portion and a length;
a handle portion associated with the drive shaft proximal end,
a fastener engaging portion associated with the drive shaft distal end, the fastener engaging portion comprising a first surface configured to axially engage a fastener and a second surface configured to rotationally engage the fastener; and
an outer sleeve associated with the drive shaft intermediate portion, the sleeve comprising a drive shaft engaging portion,
wherein the outer sleeve engaging portion and the drive shaft engaging portion are configured to coact to allow at least a portion of the drive shaft to translate linearly within the sleeve.

2. The tool of claim 1, wherein the drive shaft comprises a cannulated fastener driving portion and an inner shaft portion, at least a portion of the inner shaft portion disposed within the fastener driving portion, the inner shaft portion configured to axially engage the fastener and the driving portion configured to rotationally engage the fastener.

3. The tool of claim 2, wherein the fastener driving portion further comprises:
a driving sleeve having a distal end comprising a fastener driving end and a bore having an inner surface, and
a shaft portion comprising a distal end having a driving sleeve cooperating portion, and a cannulation for receiving the inner shaft portion of the drive shaft, wherein the distal end of the shaft portion is slidably received within the bore of the driving sleeve, and the bore and the driving sleeve cooperating portion are configured such that rotating the inner sleeve rotates the driving sleeve.

4. The tool of claim 3, wherein the inner shaft further comprises a radial groove, the shaft portion of the fastener driving portion further comprises a slot, and the driving sleeve further comprises a pin bore, wherein a pin disposed within the pin bore and extending through the slot to engage the radial groove fixes the inner shaft and the driving sleeve axially with respect to each other.

5. The tool of claim 4, wherein when the inner shaft axially engages the fastener, the driving sleeve also engages the fastener.

6. The tool of claim 1, wherein the inner shaft portion is tapered and the cannulated fastener driving portion is configured to slidingly receive the tapered inner shaft.

7. The tool of claim 1, wherein the axial fastener-engagement portion comprises a thread.

8. The tool of claim 1, wherein the first surface comprises at least one radial member configured to axially engage a recess in the head of a bone fastener.

9. The tool of claim 8, wherein the first surface comprises a plurality of radial members, each of which is configured to axially engage corresponding recesses in a fastener head.

10. The tool of claim 1, wherein the axial fastener-engagement portion grips the fastener about an outside surface of the fastener head.

11. The tool of claim 1, wherein the sleeve engaging portion and drive shaft engaging portions comprise complementary threads.

12. The tool of claim 1, further comprising an inner shaft having a fastener engaging surface at one end, the drive shaft further comprising a cannulation configured and sized to accept at least a portion of the inner shaft, wherein when the inner shaft is disposed within the cannulation the fastener engaging surface extends distally beyond the distal end of the drive shaft.

13. The tool of claim 1, wherein at least a portion of the sleeve has a roughened outer surface.

14. The tool of claim 1, the fastener engaging portion further comprising a locking clip expanding portion, the fastener disposed within a fastener hole in a plate, the fastener hole further provided with an expandable locking clip configured to engage a portion of the fastener to prevent the fastener from being backed out of the fastener hole, and wherein the locking clip expanding portion is configured to expand the locking clip.

15. The tool of claim 14, wherein the locking clip expanding portion is configured to expand the locking clip to a dimension greater than an outer diameter of the fastener head.

16. The tool of claim 14, wherein the locking clip expanding portion is configured to expand the locking clip to a dimension smaller than an outer diameter of the fastener head.

17. The tool of claim 16, wherein at least a portion of the fastener is configured to expand the locking clip to a dimension substantially equal to the outer diameter of the fastener head when the tool is engaged with the fastener and the tool is operated to remove the fastener from the bone plate.

18. The tool of claim 1, wherein the sleeve has a distal end configured to engage a bone surface.

19. The tool of claim 1, wherein the sleeve has a distal end configured to engage a surface of a bone plate.

20. The tool of claim 1, wherein the sleeve comprises first and second pieces, the first piece configured to threadably engage the sleeve engaging portion of the drive shaft and the second piece comprising an end configured to engage the surface of a bone plate or bone.

21. The tool of claim 19, wherein the first and second pieces are rotatable with respect to each other.

22. A bone plate, tool and fastener system comprising:
the tool of claim 1, further comprising at least one radial member,
a fastener having a radially deformable head and a threaded body, the head having a circumferential groove for engaging a bone plate locking element, and configured to receive the radial member to axially engage the tool with the fastener, and
a bone plate having at least one bone screw hole, the at least one bone screw hole having a locking element disposed at least partially within the hole and configured to engage at least a portion of the fastener head groove to axially retain the bone screw within the bone screw hole,
wherein when the fastener is retained within the bone screw hole by the locking element and the tool is axially engaged with the fastener, an axial removal force applied to the fastener by the tool causes the fastener head to radially deform to thereby disengage the fastener from the locking element.

23. The system of claim 22, wherein the fastener head is rendered radially compressible by at least one longitudinal slot disposed in the head.

24. The system of claim 22, wherein the fastener head is rendered radially compressible by a hollow portion disposed in the head.

25. A tool comprising
a drive shaft having a fastener engaging end and a sleeve engaging portion, the fastener engaging end comprising a rotational engagement portion and an axial engagement portion,
a sleeve disposed about at least a portion of the drive shaft, the sleeve comprising a drive shaft engaging portion,
wherein the sleeve engaging portion and the drive shaft engaging portion comprise complementary threads configured to allow the drive shaft to translate linearly within the sleeve when the drive shaft is rotated relative to the sleeve.

26. The tool of claim 25, wherein the drive shaft comprises a cannulated fastener driving portion and an inner shaft portion, at least a portion of the inner shaft disposed within the driving portion, the inner shaft portion configured to axially engage a fastener and the driving portion configured to rotationally engage the fastener.

27. The tool of claim 26, wherein the inner shaft portion is tapered and the cannulated fastener driving portion is configured to slidingly receive the tapered inner shaft.

28. The tool of claim 25, the fastener engaging end further comprising a locking clip expanding portion, the fastener engaging end of the drive shaft configured to engage a fastener disposed within a fastener hole in a plate, the plate having an expandable locking clip disposed within the fastener hole, the clip configured to engage a portion of the fastener to prevent the fastener from backing out of the fastener hole, wherein the fastener engaging end is configured to expand the fastener locking clip when the drive shaft engages the fastener.

29. The tool of claim 28, wherein the locking clip engaging portion is configured to expand the locking clip to a dimension greater than an outer diameter of the fastener head.

30. The tool of claim 28, wherein the locking clip engaging portion is configured to expand the locking clip to a dimension smaller than an outer diameter of the fastener head.

31. The tool of claim 30, wherein when the tool is engaged with the fastener and the tool is operated to remove the fastener from the bone plate, an axial removal force applied by the tool is greater than a fastener locking force of the locking clip.

32. The tool of claim 25, wherein the sleeve has a distal end configured to engage a bone surface.

33. The tool of claim 25, wherein the sleeve has a distal end configured to engage a surface of a bone plate.

34. The tool of claim 25, wherein the sleeve comprises first and second pieces, the first piece configured to threadably engage the sleeve engaging portion of the drive shaft and the second piece comprising an end configured to engage the surface of a bone plate or bone.

35. The tool of claim 34, wherein the second piece further comprises an inwardly-extending spring element configured to engage an outer surface of the drive shaft to provisionally retain the second piece at a selected location on the drive shaft.

36. The tool of claim 35, wherein the first and second pieces are rotatable with respect to each other.

37. The tool of claim 25, wherein the rotational engagement and axial engagement portions comprise a single screw thread element configured to engage and retain at least a portion of a fastener seated in bone.

38. The tool of claim 37, wherein when the tool is engaged with the fastener and the tool is rotated to remove the fastener from the bone, the rotation serves to increase engagement of the screw thread element with the fastener.

39. A method of removing a fastener from a bone and/or plate comprising the steps of:
(a) providing a tool having an inner shaft portion, a cannulated drive shaft portion and a sleeve portion, the cannulated drive shaft portion at least partially disposed within the sleeve portion and the inner shaft portion at least partially disposed within the cannulated drive shaft portion;
(b) inserting a portion of the drive shaft portion into the head of a bone fastener;
(c) axially engaging the inner shaft portion with the bone fastener, the fastener engaged with a bone portion, the fastener further disposed within the bone screw hole of a bone plate;
(d) rotationally engaging the inner shaft portion with the bone fastener;
(e) engaging one end of the sleeve portion with a surface of the bone plate; and
(f) moving the drive shaft and outer sleeve portions with respect to each other to remove the fastener from the bone.

40. The method of claim 39, wherein the inner shaft portion further comprises a threaded distal end configured to engage an internally threaded portion of the fastener.

41. The method of claim 39, wherein steps (b), (c) and (d) are performed substantially simultaneously.

42. The method of claim 39, the drive shaft portion further comprising an externally threaded portion configured to mate with an internally threaded portion of the outer sleeve, wherein step (f) comprises rotating the drive shaft and outer sleeve portions with respect to each other.
